(19) United States
(12) Patent Application Publication
(10) Pub. No.: US 2003/0004406 A1
(43) Pub. Date: Jan. 2, 2003

(54) Assembly and method for measuing ionizing radiation with background noise correction

(76) Inventors: Jean-Francois Pineau, St Sylvestre (FR); Michel Janot, Limoges (FR); Nicolas Vandeven, Uchaux (FR); Philippe Eydoux, Pierrelatte (FR)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(21) Appl. No.: 10/182,782

(22) PCT Filed: Feb. 15, 2001
(86) PCT No.: PCT/FR01/00448

(30) Foreign Application Priority Data
Feb. 16, 2000 (FR) 00/01900

(51) Int. Cl. A61B 5/05

(52) U.S. Cl. 600/407

(57) ABSTRACT

The noise originating from the ambient radiation is derived from the measurement of the radiation of a source (S) via a preliminary correlation calculated between the measurements of the main detector (1) and of at least one auxiliary detector (2): when the radiating source (S) is present, the auxiliary detector (2), which keeps on measuring the ambient radiation, is used to evaluate the proportion of the main detector (1) measurement which comes from the ambient radiation. This invention applies to the measurement of the activity of various objects proving a low radiation level.

The invention relates to the domain of measurement of ionized radiation emitted by a source, and is intended for discarding the influence of the background noise.

The specialists know that the naturally emitted nuclear radiation, received from cosmos, from ground or from the radon spread in the atmosphere cannot be neglected and may strongly affect the measurement of a low level emitting source. Another cause of error results from the occurrence of other sources which may by accident come close to the measurement site.

In the following text, the word “detector” has a general signification which may correspond to a set of several sets of detectors.

Attempts have already been made to avoid the effect of the background noise produced by the ambient radioactivity. So, it is common to have a main detector, dedicated to the measurement of the source, completed with a specific detector dedicated to background noise measurement. When the latter detects a background noise whose level is estimated as excessive, the measurement of the source is not achieved and one may wait for more adequate conditions to perform the said measurement, which may be hindering at times. According to an improved version of this system, the auxiliary detector completely surrounds the main detector and the source when it is present: it then measures the ambient radiation uniformly incoming from every direction but does not prevent the ambient radiation to cross the auxiliary detector and to reach the main detector, which still biases the measurement. This radiation may be transmitted directly or after being diffused and affected by the Compton effect, i.e. produced at a different level, which makes it difficult to evaluate from the radiation fraction that has been received by the auxiliary detector. The method consists to involve various time and space discriminations of radiation following their relative estimated directions and times of arrival on the detectors. As the auxiliary detector is also sensitive to the radiation emitted by the source, transmitted directly or after having passed through the main detector, the correcting calculations are evidently difficult to perform and require a complex electronic operating system (anti-Compton device).

The document JP 61 205886 A (Cf patent Abstracts of Japan, vol. 011 n° 035, p 542, dated Feb. 3, 1997), discloses a device which includes two detectors separated by a shield intended for measuring different radiation samples respectively originating from a reference source and from an unknown object, then for estimating the object radiation from four measurements thereof and from the reference source radiation. The background noise resulting from ambient radiation is assumed to be the same for the detectors, which moreover are confined with the reference source and the object in a shield which attenuates this noise.

The document U.S. Pat. No. 4,409,973 discloses the use of a radiation sensor in medical tomography, which involves correlation functions calculations over series of measurements. Lastly, the document U.S. Pat. No. 3,701,902 describes a geological probe and two detectors located at different distances thereof used for deducing one characteristic value of the surrounding ground from a comparison between measured values.

The object of the invention is then to discard the influence of the background noise, in the particular case of ionizing radiation measurements when it avers difficult to discriminate the influences of the various radiating source, by means of an improved detection set and an improved process. For short, the invention consists in correcting the measurements of the main detector, which must perform an evaluation of the source radiation, by estimating the background noise it detects and subtracting it from the said evaluation; it composes of a ionizing radiation measurement set, which comprises a main detector intended for measuring a radiation transmitted by a determined source (which also measures the ambient radiation) and a auxiliary detector intended for measuring an ambient radiation, characterised in that it comprises means for correlating measurements completed by the two detectors and means for correcting the measurements carried out by the main detector according to correlation results; and a ionizing radiation measurement process, including a main measurement of a main radiation emitted by a determined source and a auxiliary measurement of an ambient measurement as well as corrections of the main measurement according to the auxiliary measurement, characterised in that the main measurement includes a step of comparison between the main measurement and the auxiliary measurement in the absence of the source, the deduction of a correlation function between the main measurement and the auxiliary measurement and a step of correction of the main measurement in the presence of the source by means of the correlation function and the auxiliary measurement performed concurrently with the main measurement

These characteristics and advantages are described in the detailed description of the invention which refers to following figures:

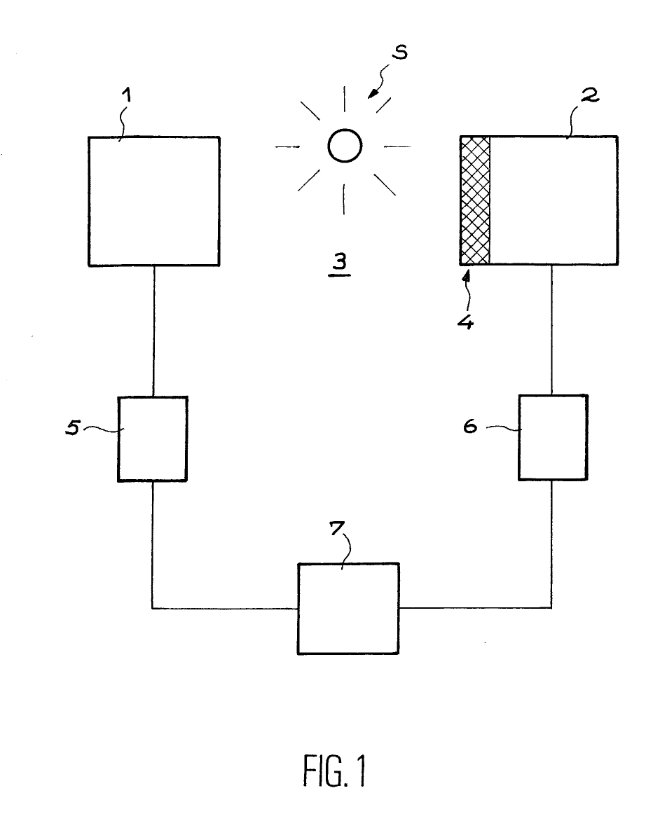

FIG. 1 is a schematic view of the detection assembly,

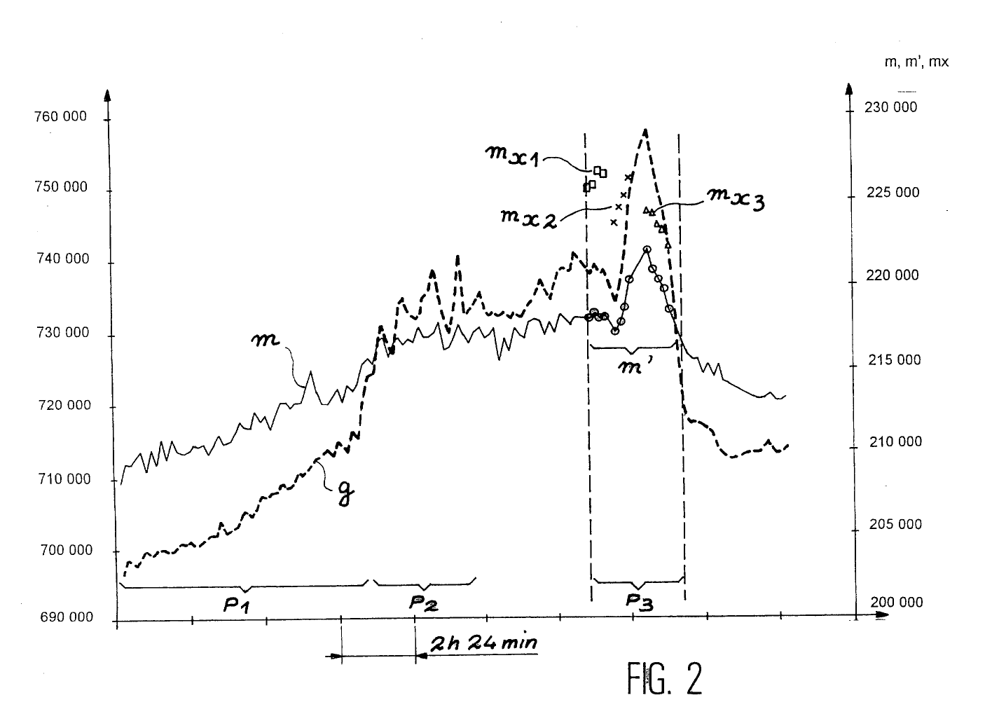

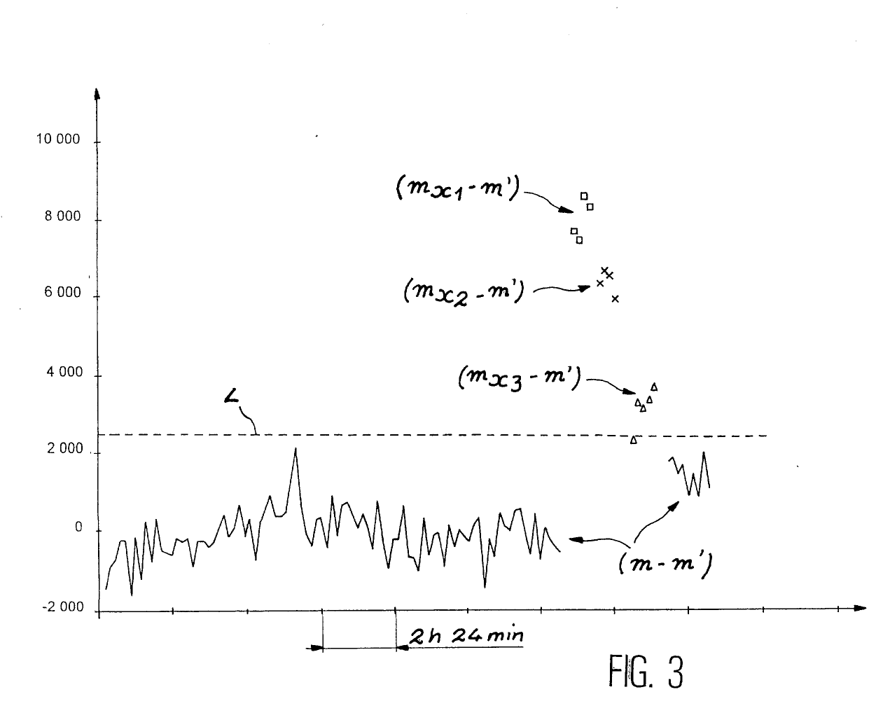

and FIGS. 2 and 3 are curves of the gross and corrected measurements.

The detection assembly according to the invention, which may take, in a particular embodiment, the form of a detection frame, is in this latter case intended for measuring the radioactivity of various objects passing along it. It includes at least a main detector, called measuring detector 1 which measures the activity of a radiating source and at least an auxiliary detector said guard detector 2, which measures the ambient radiation which causes the background noise. Detectors 1 and 2 may be located side by side, at a more or less important distance from each other, or possibly, as shown here, on both sides of a place 3 where the source S is located. The source S is any object, of any size and generally radioactive at a low level, such as a concrete block, a truck, a waste container and so on. The detectors 1 and 2, which are only sketched here, usually include an amount of radiation detectors which receive the ionizing radiation and convert them to electrical signals. The detector 1 is of course sensitive to radiation emitted in 3, whereas the guard detector 2 may be made sensitive to radiation incoming from anywhere, except from location 3, from which it is protected by a shield 4. These non-restrictive conditions allow a wide range of arrangements for detectors 1 and 2 positions. In particular, the invention is not restricted to gamma radiation and may be used for other types of radiation: alpha, beta and X, in specific configurations of the main and auxiliary detectors.

The task required from the detection assembly includes a rather short measurement duration, followed by a stand-by duration, which may reach several hours. These stand-by periods are however used for measuring the ambient radioactivity. The values measured by detectors 1 and 2 are recorded separately, then compared. The inventors discovered that measurements where correlated between one another and could be deduced from each other via a regression processing. So, the counting devices 5 and 6 to which detectors 1 and 2 are respectively linked and which are intended for concurrently recording their measurements are supplied to a computer 7 which calculates the coefficients of the correlation curve, which are in practice sufficiently approximated by a straight line.

When a radioactive source S comes to position 3 so that the detector 1 evaluates its emitted radiation, the guard detector 2 keeps on measuring the ambient radiation. The computer 7 then derives the effect of this radiation on detector 1, that is the intensity of the background noise it must estimate and subtracts it from the total measurement delivered by the counting device 5 to obtain the value of source S radiation. The results obtained so far have demonstrated the validity of this process.

Some precautions must be taken to make sure the correlation is valid. In particular, the regression coefficients somehow vary along time, so that it is advisable to use recent enough measurements only and to renew correlation calculations periodically. Moreover, all measurements are not of the same importance: the study of background noise evolution shows that it stays rather steady during long periods and slowly evolves, with no correlation between the two detectors measurements. That is why such periods of measurement fluctuations should be discarded, so as to take into account those during which measurement values continually vary along a same direction at a sufficient rate. A period of interest to perform correlation calculations is noted P1 on FIG. 2, and a low fluctuation period is noted P2.

If then the counting values from main detector 1 and guard detector 2 are M and G during a fixed data-acquisition period T, the counting rates derive from the calculation of m=M/T and g=G/T. If the correlation is linear, one may write down m′=p.g+q where p and q are the coefficients of the straight regression line and m′ is the corrected counting rate from detector 1.

During measurement periods, the measurement detector 1 counting rate will be noted mₓ and the one for detector 2 will still be noted g as this detector is shielded with respect to the location 3 of source S and stays then sensitive to the same phenomena as before. The source radiation will be derived from the corrected counting rate (mₓ−m′). It is evident that correlation laws other than linear may be considered and exploited the same way, and that some hypotheses may help to obtain the required results: the coefficient q is generally equal to 0 in a number of practical situations.

Besides the counting rates g and m over acquisition periods T of 10 minutes, FIG. 2 shows measurements mₓ₁, mₓ₂ and mₓ₃ made during an acquisition period P3 over three successive sources. These measurements where strongly affected by a noticeable increase in background noise, as the curve of the counting rate g of the guard detector 2 reached a maximum value which also appears on the background noise level m1 estimated by the measuring detector 1. Using the invention leads to the results displayed on FIG. 3, where the corrected counting rate (mₓ₁−m′), (mₓ₂−m′) and (mₓ₃−m′) are plotted, as well as the background noise processed by the correlation, equal to (m−m′). The latter is by far lower than m, whereas the corrected counting rates are more coherent than the raw rates mₓ₁, mₓ₂ and mₓ₃ for each one of the three sources, and certainly close to the ideal values one should have found, though the background noise was huge in this case, that is more than 20 times the signal value. The curve L delineates the detection limit under which one may not be able to correctly measure any radiation level: the “reduced” noise stays always under this limit and the results seem to be correct even for the third source, whose radioactivity is scarcely above this limit.

An important advantage of the invention is that the range of radiation intensities liable to be measured is actually increased.

To conclude, the invention not only facilitates the detection of radiation but also facilitates the measurement thereof; it may then apply to other devices than detection frames.

1. Ionizing radiation measurement assembly, at least including a main detector (1) intended for measuring a radiation emitted by a determined source (S) and also measuring an ambient radiation, and at least one auxiliary detector (2) intended for measuring the ambient radiation only, characterised in that it includes means (7) for calculating a correlation function of measurements of the ambient radiation carried out by the two detectors and for correcting the measurements completed by the main detector according to results of the correlation.

2. Ionizing radiation measurement assembly according to claim 1, wherein the detectors are located on both sides of a location (3) intended for placing the source or side by side near this location, the detector (2) being protected by a shield (4) from this location.

3. Ionizing radiation measurement process including a main measurement of a main radiation (m) emitted by a given source (S) and an auxiliary measurement (g) of an ambient radiation, as well as corrections of the main measurement according to the auxiliary measurement, wherein the main measurement includes a step of comparison between the main measurement and the auxiliary measurement while the source is not present, an evaluation of a correlation function between the main measurement and the auxiliary measurement, and a step of correction of the main measurement while the source is present using the correlation function and the auxiliary measurement performed concurrently with the main measurement.

4. Ionizing radiation measurement process according to claim 3, wherein the measurements are compared at a series of times within a determined lapse of time.

5. Ionizing radiation measurement process according to claim 3, wherein the measurements are compared during continuous variation periods of measurements.
